(19) United States
(12) Patent Application Publication
(10) Pub. No.: US 2024/0185954 A1
(43) Pub. Date: Jun. 6, 2024

(54) PREDICTIVE METHOD FOR DETERMINING THE PATHOGENICITY OF COMBINATIONS OF DIGENIC OR OLIGOGENIC VARIANTS

(71) Applicant: ENGENOME S.R.L., Pavia (IT)

(72) Inventors: Ivan LIMONGELLI, Pavia (IT); Susanna ZUCCA, Pavia (IT); Federica DE PAOLI, Pavia (IT); Ettore RIZZO, Pavia (IT); Paolo MAGNI, Pavia (IT); Federica BACCALINI, Pavia (IT)

(21) Appl. No.: 18/550,662

(22) PCT Filed: Mar. 16, 2022
(86) PCT No.: PCT/IB2022/052386

§ 371 Date: Sep. 14, 2023

(30) Foreign Application Priority Data
Mar. 17, 2021 (IT) 102021000006353

(51) Int. Cl. G16B 25/10; G16B 20/20; G16B 20/50

(52) U.S. Cl. CPC G16B 25/10; G16B 20/20; G16B 20/50

(57) ABSTRACT

A computer-implemented method determines pathogenicity of combinations of digenic or oligogenic variants related to a disease. A set of variants is defined and the pathogenicity is determined. The variants refer to mutations in one/both alleles of one of at least two genes. Each gene is associated with two alleles. Presence or absence of the variants in the alleles of the genes is determined. Each situation is associated with a respective combination in which each variant is present in a subset of alleles. For each combination and gene, a pathogenicity index/score is calculated, to estimate how much the variants modify functioning of the gene. The method includes describing phenotypic traits of a patient, by standardized information for patient phenotypic abnormalities, and providing input information for the pathogenicity determination to a trained algorithm. Output information is obtained from trained algorithm, representing pathogenicity of each combination of variants/mutations.

### CROSS-REFERENCE TO THE RELATED APPLICATIONS

The present application is a National Stage Filing of PCT International Application No. PCT/IB2022/052386 filed on Mar. 16, 2022, which claims priority to Italian Patent Application No. 102021000006353 filed on Mar. 17, 2021, both of which applications are incorporated herein by reference in their entirety.

### FIELD OF APPLICATION

The present invention relates to a predictive method for determining the pathogenicity of combinations of digenic or oligogenic variants.

Therefore, the general technical field of the present invention is that of predictive methods, performed by electronic computation, used in the context of genomics and/or medical genetic research to support predictive prognoses.

### DESCRIPTION OF THE PRIOR ART

For decades, the mechanism of inheritance of a genetic disease has been explained through the reductive paradigm “one gene, one disease” according to which mutations (otherwise referred to as variants) which compromise a single gene turn out to be causative of several rare diseases of Mendelian inheritance.

More complex genetic models have been developed in recent years to explain a series of genetic disorders the causation of which cannot be solved through the mutation on a single gene (see for example, Katsanis N. “The continuum of causality in human genetic disorders”. Genome Biol. 2016; 17(1):233. https://doi:10.1186/s13059-016-1107-9).

Some of these pathological disorders require simultaneous multiple mutated genes to develop the symptoms of the disease while others are characterized by a primary causative gene and a boundary gene network which has the role of modulating the outcome of the disease, the severity of the symptoms or the onset thereof.

Recently, some machine learning-based models have been developed to discriminate combinations of digenic variants into pathogenic or benign variants. In this respect, reference should be made, for example, to the following scientific papers:

Papadimitriou S., Gazzo A., Versbraegen N., Nachtegael C., Aerts J., Moreau Y., Van Dooren S., Nowe A., Smits G., Lenaerts T., “Predicting disease-causing variant combinations”. Proc. Natl. Acad. Sci. U.S.A. 2019; 116(24), 11878-11887. https://doi.org/10.1073/pnas.1815601116

Mukherjee S., Cogan J., Newman J., Phillips J., Hamid R., Meilerì J. Capra, J., “Identifying digenic disease genes using machine learning in the undiagnosed diseases network”, Preprint at bioRxiv. 2020.05.31.125716.

doi: https://doi.org/10.1101/2020.05.31.125716

Renaux A., Papadimitriou S., Versbraegen N., Nachtegael C., Boutry S., Nowé A., Smits G., Lenaerts T. “ORVAL: a novel platform for the prediction and exploration of disease-causing oligogenic variant combinations”. Nucleic Acids Res. 2019 Jul. 2; 47(W1):W93-W98. https://doi:10.1093/nar/gkz437.

However, these approaches enrich each variant individually with molecular, pathological or predicted effect information and integrate the digenic interaction information, seeking evolution patterns and biological pathways shared by the two genes involved.

The main limitation of these methods is represented by not at all considering the information related to the patient's phenotype, thus neglecting an aspect which can provide very useful information for accurately identifying causative variants.

### SUMMARY OF THE INVENTION

It is the object of the present invention to provide a predictive method, implemented by computer processing, to determine the pathogenicity of combinations of digenic or oligogenic variants, which allows solving, at least partially, the draw backs described above with reference to the prior art, and responding to the aforesaid needs particularly felt in the technical field considered.

### BRIEF DESCRIPTION OF THE DRAWINGS

Further features and advantages of the method according to the invention will become apparent from the following description of preferred exemplary embodiments, given by way of non-limiting indication, with reference to the accompanying drawings, in which:

FIG. 1 depicts a simplified block diagram showing an embodiment of the method according to the invention;

FIG. 2 depicts a block diagram of a training procedure of a trained algorithm, included in an embodiment of the method according to the invention;

FIGS. 3A-3F show some examples of possible digenic variants.

### DETAILED DESCRIPTION

A predictive method is described, implemented by computerized processing, to determine the pathogenicity of combinations of digenic or oligogenic variants, in relation to a disease.

The method firstly comprises a step of defining a set of variants, the pathogenicity of which must be determined. Such variants refer to mutations present in one or both alleles of a respective gene of at least two genes, in which each of the genes is associated with two respective alleles.

The method then includes determining the situations which can occur, regarding the presence or absence of the aforesaid variants in the alleles of the at least two genes considered. Each situation is associated with a respective combination in which each variant is present in a respective subset of alleles, among all the possible subsets of alleles of all the genes considered, or in which the variant is present in all the alleles of all the genes considered.

For each of the defined situations, i.e., for each combination and for each gene, the method includes then calculating a pathogenicity index or score, adapted to estimate how much the one or more respective variants modify the functioning of the respective gene.

The method further comprises the steps of describing phenotypic traits of a patient, by standardized phenotypic terms, i.e., standardized information adapted to describe phenotypic anomalies found in the patient, and calculating or preparing input information for the pathogenicity determination.

Such input information for the pathogenicity determination comprises the following four types of features:
gene-phenotype association features, calculated individually for each of the genes considered, and adapted to measure how much the aforesaid phenotypic traits of the patient are superimposable to phenotypes already known to be associated with the single gene;
digenicity or oligogenicity features, calculated for each of the aforesaid combinations of genes, adapted to capture the interaction between the genes forming each combination;
a priori property features of the genes, calculated for each of the aforesaid genes considered;
variant-related features (i.e., features at variant level), calculated, for each gene considered, based on the aforesaid pathogenicity indices or scores calculated in relation to all the combinations considered.

The method further includes providing said input information for the pathogenicity determination to at least one trained algorithm, and processing said input information for the pathogenicity determination by the at least one trained algorithm.

The trained algorithm is an algorithm trained by artificial intelligence and/or machine learning techniques.

In particular, the aforesaid algorithm is trained in a preliminary training step, based on a training dataset of known cases, providing the aforesaid input information calculated for each of the known cases to the algorithm to be trained, and training the algorithm based on the knowledge of the pathogenicity/benignity of the respective known cases.

Finally, the method comprises the step of obtaining output information from the trained algorithm, representing the pathogenicity of each of the combinations of variants or mutations considered.

According to an embodiment, the method is adapted to determine the pathogenicity of digenic variants.

In such a case, in the aforesaid step of determining possible situations, the identifiable situations comprise, for each of the variants considered and for each of the two genes, a situation of simple heterozygosity, in which the variant is present in only one of the two alleles, and a situation of compound heterozygosity, in which two different variants are present and each of the two variants is present on a respective allele, and a further combination of homozygosity, in which the same variant is present in both alleles of the gene.

Furthermore, in this case, the aforesaid gene-phenotype association features comprise four features, two for each of the two genes; the aforesaid features of digenicity or oligogenicity comprise two features of digenicity calculated with reference to the two genes; the aforesaid features of a priori properties of the genes comprise six features, three for each gene; and the aforesaid features at variant level comprise two features, one for each of the two genes, calculated for each gene considered as a combination of the pathogenicity scores of the variant(s) which refer to said gene.

To calculate such a combination of scores, in this implementation example, the method uses the sum of the scores assigned individually to each variant which falls on one of the two alleles of the same gene.

According to another embodiment, the method is adapted to determine the pathogenicity of oligogenic variants, referring to N genes.

In such a case, the aforesaid gene-phenotype association features comprise 2N features, two for each of the N genes; the aforesaid digenicity or oligogenicity features comprise two oligogenicity features calculated with reference to the N genes; the aforesaid a priori property features of the genes comprise 3N features, three for each gene; the aforesaid features at variant level comprise N features, one for each of the N genes, calculated for each gene considered as a combination of the pathogenicity indices or scores of the variant(s) which refer to said gene.

In accordance with an embodiment of the method, the step of describing phenotypic traits of a patient comprises describing phenotypic traits through terms deriving from an ontology which provides a standardized vocabulary of phenotypic anomalies found in human diseases, or preferably through the HPO terms, deriving from the resource Human Phenotype Ontology.

According to an implementation option, the description of phenotypic traits by HPO is represented by a direct acyclic graph.

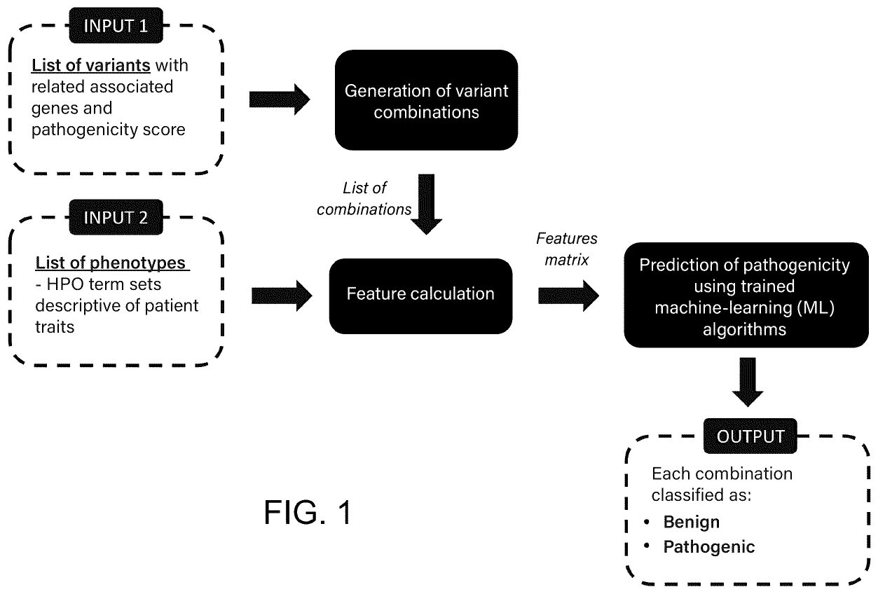

In accordance with an embodiment (shown for example in FIG. 1), the method comprises, after the step of calculating, a pathogenicity index or score for each variant identified in the patient, and after the step of describing a patient's phenotypic traits, the following further steps:
providing a list of defined variants, with the respective genes and respective calculated pathogenicity indices, to a first pre-processing algorithm, configured to generate a list of possible combinations;
processing such a list of possible combinations, generated by the first pre-processing algorithm, as well as the aforesaid phenotypic traits of the patient, in the terms described, by a second pre-processing algorithm, to calculate or prepare input information for the pathogenicity determination.

According to various possible implementation options, the aforesaid first pre-processing algorithm and second pre-processing algorithm (represented by separate blocks in FIG. 1) can be implemented by any combination of one or more software programs or modules, executable by a computer or processor.

In accordance with a method embodiment, said gene-phenotype association features comprise, for each of the genes considered, an index or measure of similarity between the set of standardized phenotypic terms describing the patient and the set of standardized phenotypic terms associated with the gene, and a probability of association between the single mutated gene and the set of phenotypes describing the patient using gene expression data, or starting from a transcriptomics analysis.

According to an implementation option, by means of the aforesaid index or measure of similarity, the method captures the similarity between the set of phenotypes characterizing the patient and the phenotypes which have been found, according to the resources of the Human Phenotype Ontology (not necessarily all simultaneously), in individuals having such a mutated gene. The list of genes used to calculate such a feature is inferred from the genes affected by the variants of the individual patient.

According to an implementation example, in which the phenotypes are organized within a direct acyclic graph, the similarity of the pairs of phenotypes is calculated based on the proximity thereof within the graph. Once the measure of pairwise similarity between each phenotype of the patient and each corresponding phenotype associated with the gene has been calculated, the similarity measures are combined, using standard methodologies known per se, with the aim of providing a single measure which expresses the similarity between two sets of phenotypes.

According to an implementation option, the formulation of the above association probability (i.e., gene-phenotype association index) includes calculating an association score of each patient phenotype with each mutated gene which is then normalized on all patient phenotypes to obtain the final association score. The advantage of this index is that by using transcriptomics data (i.e., gene expression data), the prediction is based on mechanisms of co-regulation between genes annotated by a certain HPO term with others (e.g., genes with a correlation in terms of gene expression are likely to be involved in the same biological mechanism, and thus if one thereof is annotated by a certain phenotype, it is likely that co-regulated genes are also somehow associated with such a phenotype). This also allows defining a priority of those little-known genes which do not have a phenotypic characterization known in the literature, thus providing complementary information to that provided by the similarity index mentioned above.

In accordance with a method embodiment, the aforesaid digenicity or oligogenicity features, for each combination of genes, comprise a biological distance measure or index and a similarity measure or index between the phenotypic sets associated with the genes, regardless of the phenotypic traits describing the patient.

The measure or index of biological distance represents how much the proteins produced by the two genes are involved or not involved in the same biological processes with a certain degree of interaction, or the measure or index of biological distance represents the degree of functional association between two or more genes, articulated according to a series of levels of evidence.

In accordance with a method embodiment, the biological distance is a measure which captures the degree of functional association between two or more genes, capable of describing how much the aforesaid genes are involved in the same biological processes, according to different levels of evidence (for example: experimental activity or analysis of scientific literature). The biological distance value decreases as the association which exists between the two genes studied increases and can also be expressed in the form of complementary probability.

In accordance with a possible method implementation, the phenotypic similarity index is a measure which captures the degree of similarity between the two genes of the pair in terms of phenotypic associations of each gene, regardless of the phenotypes associated with the sample. Such a measure is based on the comparison of the two phenotypic sets associated with each gene to define how superimposable they are (for example, by exploiting the structure of the resource Human Phenotype Ontology, representable through an acyclic graph).

In accordance with a method embodiment, the aforesaid a priori property features of the genes comprise, for each gene, one or more measures or indices representing how sensitive each gene is or is not to the gene dosage.

According to an embodiment, the method comprises, before using the aforesaid trained algorithms, a further preliminary training step, performed based on the two subsets of said training dataset containing data referring to known cases: a first subset which is used as a training database, or “training set”, and a second subset which is used as a validation database or “test set”.

According to an implementation option, the preliminary training step comprises training a plurality of trained algorithms, or classifiers, and evaluating the performance of each classifier.

According to another implementation, the method further comprises the step of selecting a subset of trained algorithms, for the processing, based on the aforesaid preliminary performance evaluation.

In accordance with a method embodiment, once the preliminary training step has been completed, the step of processing the input information for the pathogenicity determination comprises processing the information by all the trained algorithms or classifiers used in the training, or by the trained algorithms or classifiers selected during the training.

In accordance with various possible method implementation options, the aforesaid trained algorithms or classifiers comprise one or more of the following algorithms or techniques: Random Forest, AdaBoost, Gradient Boosting, Logistic Regression, Multi-layer Perceptron, Decision Tree.

According to an implementation option, the aforesaid first training subset, or “training set”, is generated from two data resources: a first data resource comprising examples of positive (i.e., pathogenic) inputs, which represent combinations of digenic variants known to be causative of a digenic disease, in turn characterized by a specific set of phenotypes; and a second data resource comprising examples of negative (i.e., benign) inputs, consisting of combinations of digenic variants known to not be causative of disease.

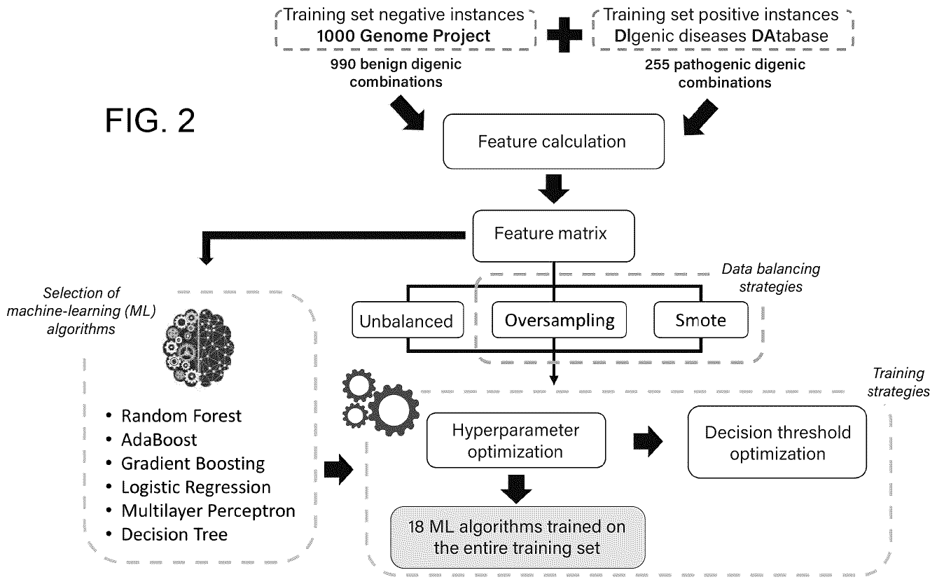

According to a particular implementation example (shown at the top of FIG. 2), the aforesaid first data resource is obtained from the 1000 Genome Project database and the aforesaid second data resource is obtained from the Digenic Disease Database (DIDA).

In the particular training example shown in FIG. 2, 990 digenic combinations from the 1000 Genome Project database, for which it has been ascertained that they do not cause a specific set of phenotypes, are used for training; 255 digenic combinations from the DIDA database, recognized as causing a digenic disease characterized by a specific set of phenotypes, are used for training.

According to an implementation option (also shown in FIG. 2) the method comprises the further step of balancing the data, in the case of data resources unbalanced towards negative cases, to obtain a balanced distribution between the two classes. In several possible implementation examples, this step of balancing the data is carried out by the “oversampling” methodology or by SMOTE methodology.

Therefore, as shown in FIG. 2, downstream of the features matrix, and before the training strategies, different method implementation options include three respective, mutually alternative possibilities: maintaining unbalanced data (if the unbalanced conditions are deemed acceptable), or balancing the data by oversampling technique or balancing the data by SMOTE technique.

In accordance with a method embodiment, the aforesaid output information comprises an estimated pathogenicity probability of at least one combination of digenic or oligogenic variants considered, or of a plurality of combinations of variants among the digenic or oligogenic variants considered, or of all the combinations of digenic or oligogenic variants considered.

According to an implementation option, the output information further comprises, for each combination of digenic or oligogenic variants, a binary result representative of whether the combination of digenic or oligogenic variants is “pathogenic” or “benign,” obtained by comparing the pathogenic probability estimated for the digenic or oligogenic variant with a respective threshold, associated with the combination itself of digenic or oligogenic variants.

According to a method embodiment, the aforesaid output information comprises identifying the most relevant oligogenic combination of variants, or the best digenic pair of variants, from a set of combinations of oligogenic or digenic variants considered, having the most relevant correlation with the set of phenotypes considered.

Further details will be provided below, by way of non-limiting example, referring to some particular embodiments of the method.

As shown above, the approach used in the present method is based on machine-learning techniques and further integrates patient phenotype information, in order to improve accuracy in the classification of oligogenic, and in particular digenic, variants. The patient's phenotypic traits are described through the HPO terms, deriving from the resource Human Phenotype Ontology, which is an ontology that provides a standardized vocabulary of the phenotypic anomalies found in human diseases.

The Applicant has also developed a series of models with the aim of evaluating different machine-learning algorithms adapted to solve the problem, at the same time verifying whether the features chosen allowed describing all the aspects of a combination of digenic variants and the related associations with the patient's phenotypic traits.

As already described above, a first step of the method provides grouping the identified variants into one sample per gene and generates all the possible combinations.

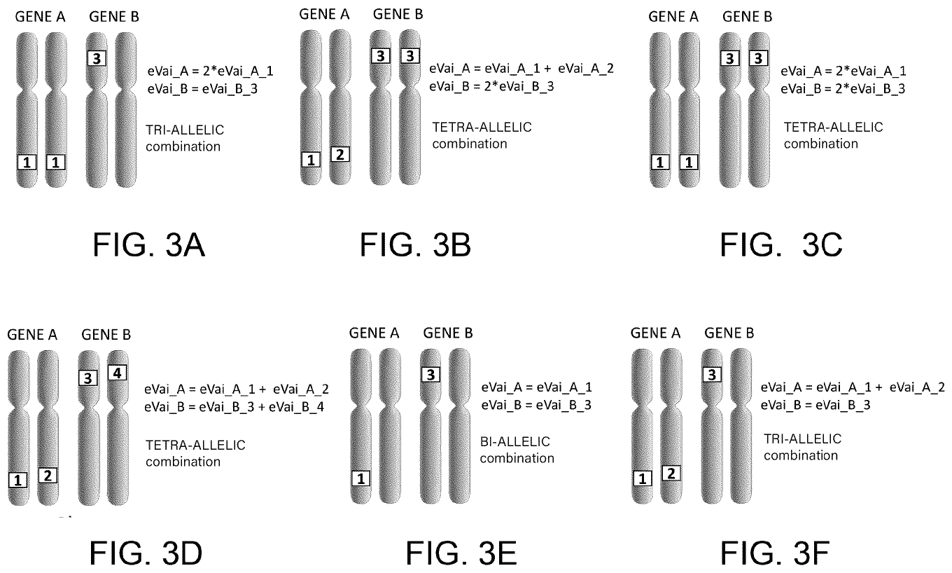

Assuming that the genes involved in a digenic syndrome are gene A and gene B (as shown in FIG. 3), and knowing that each of them is present in the human genome in duplicate (two alleles), for each gene the mutation can be absent (zero mutated alleles), or present in only one of the two alleles (heterozygosity) or present on both alleles of the same gene (homozygosity).

It follows that the total number of mutated alleles for the pair of genes gene A and gene B ranges from zero to four and that reference is made to a digenic combination when at least two alleles (one for gene A and one for gene B, as in FIG. 3E) are carriers of mutation.

Depending on the total number of mutated (or mutant) alleles, combinations of di-allelic (FIG. 3E), tri-allelic (FIGS. 3A and 3F) or tetra-allelic (FIGS. 3B, 3C and 3D) variants are identified.

More specifically: FIG. 3A) Homozygous variant on gene A+simple heterozygous variant on gene B. FIG. 3B) Two heterozygous variants composed on gene A+homozygous variant on gene B. FIG. 3C) Homozygous variant on gene A+homozygous variant on gene B. FIG. 3D) Two heterozygous variants composed on gene A+two heterozygous variants composed on gene B. FIG. 3E) Simple heterozygous variant on gene A+simple heterozygous variant on gene B. FIG. 3F) Two heterozygous variants composed on gene A+simple heterozygous variant on gene B.

The method provides that, for each combination, a pathogenicity score (referred to as “eVAI” in FIG. 3) is calculated for each gene, wherein the pathogenicity score also takes into account the zygosity of the variants, as shown in FIG. 3.

Some implementation options of the method include calculating the pathogenicity score by an algorithm which is proprietary to enGenome (eVai pathogenicity score) or by other methodologies which are known per se (e.g., CADD score). Such an aspect will be further elaborated hereinafter.

The examples to be provided as an input to the machine learning model are thus the combinations of candidate variant being causative in a digenic model with the characterization of the phenotype associated with the sample.

The features of the model (i.e., the attributes which express the knowledge available on each pair of candidate variants) have been defined a priori with the aim of capturing all the aspects of a digenic combination; for such a reason, all the features described below are useful to describe the combination of variants and each of them has a dedicated module within the algorithm.

In the detailed example illustrated here, there are 14 such features in total and they can be divided into 4 macro-categories or groups, three of which represent properties of the single gene forming the pair (hereinafter referred to as 1, 3 and 4) while one captures the digenic interaction (hereinafter referred to as 2).

### Features Group 1: Gene-Phenotype Association Features

These features are calculated individually for the two genes involved in the combination and measure how much the patient's phenotypes are superimposable to phenotypes already known to be associated with the single gene. In the example considered here, 4 total features belong to this group, 2 for each gene.

The association of the patient's phenotypes with those related to the single gene is expressed according to two different methodologies:
a. As a similarity between the set of HPO terms describing the patient and the set of HPO terms associated with the gene: this can be calculated through a set of standard methodologies which combine the measures of similarity of pairs of HPO terms, such as the Resnik and Lin measures. In this respect, the HPOSim package, developed in 2015 at the Xidian University School of Technology and Computer Science, can be used to analyze the phenotypic similarity to genes and diseases, based on human ontology data. By incorporating such a package into the operating flow of the method, it is possible to calculate a phenotypic similarity score which represents how much each gene of the pair is associated with the phenotypes manifested by the individual; considering two genes, there are two association scores, referred to as HPOSim A and HPOSim B, respectively.
b. As association probability between the single mutated gene and the set of phenotypes which describes the patient using gene expression data, thus starting from a transcriptomics analysis. For example, the Gado (GeneNetwork Assisted Diagnostic Optimization) feature can be used, which takes its name from the integrated tool used to calculate the feature itself (Gado is a software developed in Java language in 2018 by the Department of Genetics from the University Medical Center of Groningen). In the digenic model, there are two Gado features: Gado A and Gado B, each of which expresses the association of the individual gene with the phenotypic set of the sample.

### Features Group 2: Digenicity (or Oligogenicity) Features

These features express the interaction between the genes forming each combination and thus represent the “digenic” component of the model. The interaction can be expressed according to different aspects: (a) biological distance; (b) measure of similarity between the phenotypic sets associated with the genes.
a. The “biological distance” represents how much the proteins produced by the two genes are involved or not involved in the same biological processes with a certain degree of interaction; such a measure thus captures the degree of functional association between two or more genes, articulated according to a series of levels of evidence (co-expression data, experiments, text-mining, . . . ).

In this respect, it should be noted that biological networks offer the possibility of studying biological phenomena from a systemic point of view in which the object of the study is the interaction between the components of the network. The network described here is the interactome, i.e., the set of protein-protein interactions (PPI) occurring in human cells. A network is itself a discrete object represented by a graph. The graph consists of arcs and nodes: G=(Vertex, Edge), the arcs describe the relationships which exist between the nodes. A network is also a topological structure on which a metric can be defined, a measure of distance determined as the number of arcs separating the nodes.

The graph representing the human interactome was generated using the protein database STRING, Search Tool for the Retrieval of Interacting Genes/Proteins. In particular, this is a non-oriented weighted graph, in which each node is a protein and each arc is assigned a weight which represents the probability of interaction between two vertices. The biological distance is thus a correlation metric between two (or more) proteins based on protein-protein interaction information. STRING provides the association scores for each pair of proteins.

There are seven types of scores, related to different types of tests, referred to as evidence channels:
proximity, co-occurrence, fusion are three prediction channels based on information on the genomic context, resulting from systematic genomic comparisons aimed at evaluating evolutionary events of gene gain, loss or fusion, since such non-random gene arrangements with respect to gene function allow the inference of functional associations between genes;
co-expression, based on gene correlation tests obtained by processing RNA-Seq samples;
experiments performed on organisms from which biochemical and genetic data have been obtained;
databases related to information on biological pathways of various organisms (KEGG, Reactome, Gene Ontology);
text-mining, i.e., data from automated literature analysis, collecting evidence related to biochemical and genetic data.

Finally, the last score, “combined score”, used in the present example of the method, is calculated by integrating the probabilities of the various channels, and correcting the probability of randomly observing an interaction. This score is often higher than the individual sub-scores, expressing greater confidence when an association is supported by different types of evidence. It is calculated based on the assumption of independence between the various sources, by means of the Naive Bayes method. Therefore, it is a simple expression of the individual scores Si:

STRING_{score}=1−Π(1−Sᵢ)

Such a value represents the association probability between the single pairs of proteins. The weight attributed to the conjunction arc between two gene products is calculated with the following expression:

$$weight = 1 - \frac{{STRING}_{score}}{1000}$$

because a measure of proximity represented by the complementary probability is desired. The probability value is reported in the canonical range [0, 1].

Starting from the database, the biological network is precomputed (for example, through the Python NetworkX library). When creating the graph, the adjacency matrix is also calculated, from which the maximum distance between two nodes is obtained; such a value is necessary to attribute a distance value in the event that two nodes are so far away (or not belonging to a single graph) to prevent the algorithm from efficiently determining the value thereof and is assigned as follows:

cutoff=max_distance+(max_distance/2)

The distance between two objects in a graph is the typical problem of finding the shortest path, which can be solved by means of dynamic programming techniques, in which the problem is divided into sub-problems and optimal substructures are used. The algorithm chosen to perform the operation is the Dijkstra algorithm available in NetworkX. Once the source protein and the target protein have been indicated, such an algorithm searches for the minimum path of conjunction between the two elements within the graph. The complementary probability has been assigned to the weight, precisely because the optimal path is represented by the path which minimizes the sum of the weights. For each combination of proteins associated with the two genes, the optimal path is calculated by means of the Dijkstra algorithm. A list of distances between the pairs of proteins of the two genes is thus obtained: the biological distance is the minimum value of the list.
b. A measure of similarity between the phenotypic sets associated with the genes, regardless of which traits describe the patient. Like feature 1a), this measure on an ontological basis can also be expressed according to different methodologies.

For example, still using HPOSim, a score can be calculated which represents a measure of gene interaction based on ontological information, thus the score refers to the pair of genes regardless of the phenotypes describing the sample (referred to as HPOSim AB). For the calculation of such a value, the Lin coefficient defined as follows was used as a measure of semantic similarity:

$$Lin = 2 * \frac{IC ( {p}_{MICA} )}{IC ( {term}_{1} ) * IC ( {term}_{2} )}$$

in which:
p_{MICA} represents the most informative common ancestor, i.e., that with the least genes associated between the two terms the similarity of which is calculated;
term₁ and term₂ are the two terms the similarity of which is to be calculated.

In HPOSim, the similarity between two genes is calculated based on the pairwise similarity of the two sets of HPO terms which annotate the genes themselves. HPOSim provides 5 methods to combine multiple measures of term similarity into a single gene-gene score. In the present method example, the best match average (BMA) method was chosen, represented by the equation:

$${Sim}_{BMA} ( {g}_{1} , {g}_{2} ) = \frac{{\sum }_{i = 1}^{m} {max}_{1 \leq j \leq n} {s}_{ij} + {\sum }_{j = 1}^{n} {max}_{1 \leq i \leq m} {s}_{ij}}{m + n}$$

in which:
s represents the similarity value between the two HPO terms i and j;
m and n are the number of terms annotated to gene A and gene B, respectively.

The association scores, calculated with Lin measurement in pairs for the two phenotypic sets, are combined into a single score using the BMA method. The digenic feature HPOSim AB is calculated using the set of terms of which the two genes are annotation, considering not only the information content of the most common ancestor, but also the information related to each term (Lin). This is performed through the designated HPOSim functionality (getGeneSim); it is based on the navigation of the ontological tree, for the search for the common ancestor and the calculation of the intrinsic information measure.

### Features Group 3: A Priori Gene Properties

These features, in the present example, are 6 in total, 3 for each gene inserted in the combination. They are independent of both the variant(s) associated with the single gene (i.e., the value of the feature does not depend on which specific variant is present on the gene, but only on whether the gene is mutated or not), and the phenotypes which characterize the patient. Such features are divided into measures which capture how sensitive each gene is or is not to the gene dosage; for example, haploinsufficiency indicates in which cases a single wild-type copy (not mutated) of the gene is insufficient for performing normal biological functions.

In the method embodiment shown herein, such features are “Haploinsufficiency”, “Recessivity” and “Insensitivity to gene dosage”.

Haploinsufficiency is the condition whereby the presence of a single wild type allele is insufficient for the correct functioning of the associated protein. In particular, if the healthy allele is not overexpressed, only 50% of the gene product will be synthesized, an insufficient amount for performing the biological functions.

Haploinsufficiency is also indicated as a condition of sensitivity to gene dosage. Such a property is typically causative of an autosomal dominant disease. Geneticists have identified over 300 of these genes, but it is not known how many of the 22,000 genes may be sensitive to the loss of function of an allele.

Rather, recessivity is the condition whereby the gene is associated with an autosomal recessive condition. In this case both copies of the gene must be affected by a “loss of function” mutation to be causative of the phenotype while in the presence of a single mutated allele the disease does not occur.

The Clinical Genome Resource, ClinGen, is a project funded by NIH with the aim of building a knowledge base on genes and variants of clinical relevance, to improve research and care in precision medicine. The projects carried out and made available by ClinGen include gene classification work related to dosage sensitivity; in fact, the consortium has developed an evaluation system which lists the tests supporting haploinsufficiency for individual genes and genomic regions, following certain criteria including the number of causative mutations reported and the tests from large-scale case-control studies. The system is designed to be dynamic in nature, with regions being periodically re-evaluated to incorporate emerging evidence. When evidence for a gene is available, it is assigned one of the following 5 scores:
3: sufficient evidence to support dosage sensitivity. The tests necessary for such cataloguing are loss of function mutations in at least three unrelated probands and with similar phenotypes. The information must come from at least two independent publications.
2: emerging dosage sensitivity tests. Two probands with causative mutations of the phenotype are necessary or alternatively they can be mutations emerging from case-control studies, albeit with undefined phenotype.
1: little evidence. When only one causative loss of function mutation of phenotypes has been highlighted.
0: no evidence. No loss of function mutation was detected in subjects with clinical phenotype.
40: unlikely dosage sensitivity. Classification applied when there is evidence to support the hypothesis that the genomic region is not associated with haploinsufficiency.
30: gene associated with autosomal recessive phenotype. When the phenotype is caused by a mutation with recessive genotype.

Using the 5 classification levels provided by ClinGen, 3 types of single-gene features are created:
Rec, refers to the recessivity of the gene. It is a binary feature which assumes a value of 1 when the gene is recessive, 0 when it is haploinsufficient or not sensitive to dosage.
Haplo, indicates haploinsufficiency and is distributed over 5 values: 0, 1, 2, 3 corresponding to the four evidence levels of ClinGen, −1 when the gene is recessive or has been shown to be non-sensitive to dosage.
Dos, expresses insensitivity to dosage; assumes a value of 1 when there is evidence of such a condition corresponding to 40 of ClinGen, 0 if the gene is recessive or if it is haploinsufficient.

In analogy to the other nomenclatures, there will be: Haplo A, Haplo B, Dos A, Dos B, Rec A, Rec B. Because they contain a priori information about the gene, such features are independent of both the specific mutation needed on the gene and the phenotypes of the sample.

### Features Group 4. Variant-Related (i.e., Variant-Level) Features

These features are calculated separately for each gene of the pair and represents a combination of the pathogenicity scores of the variant(s) which fall on that gene and form the combination.

The pathogenicity score, which captures how much the variant impacts the functioning of the gene, can be calculated with different methodologies (e.g., eVAI pathogenicity score, CADD score, . . . ).

In the implementation option shown here in detail, the “eVai” score is used to calculate this feature.

The term “eVai” is referred to as a software predictor of pathogenicity of rare monogenic variants developed in 2016 by the enGenome team. The “eVai” feature also takes its name from the predictor software with which it is calculated. eVai, following the ACMG guidelines, can classify a variant into five categories:
benign
likely benign
uncertain significance (VUS)
likely pathogenic
pathogenic

eVai elaborates a file containing the mutations of a subject, typically obtained from genome sequencing with NGS technique, and evaluates each variant according to one or more phenotypic conditions associated therewith. The software returns a file containing a series of annotations, some of which are used as algorithm input to construct the variant combinations:
information identifying the variant, such as chromosome, start position, wild type reference base, mutated base;
HGNC gene affected by the mutation;
phenotypic condition according to which the variant is classified;
genotype of the variant;
classification into one of the five categories;
pathogenicity score.

Starting from the eVai output file, the combinations of mutations are composed and the score of the variant present on the gene is reported. Therefore, the eVai score, which represents the calculated pathogenicity score for each individual variant, is located in the input file provided to the module for the calculation of the features. Such a module extracts the values of eVai A and eVai B from the input file calculated in the previous step.

This is a feature of the single gene category which considers the variants which impact the functionality thereof, as there are two eVai scores one for each gene in the instance. The genotype provides information on the zygosity of the variant and influences the calculation of the eVai features. In fact, if the genotype is heterozygous or hemizygous, the eVai feature is equal to the value of the eVai pathogenicity score. If both alleles are mutated, the pathogenicity feature considers both eVai scores. Therefore, in the case of homozygosity, there will be twice the score of the single variant.

In the case of compound heterozygosity, the “eVai” score is the result of the sum of the two pathogenicity scores deriving from the individual variants.

eVai_{eterozigote}=eVaiₐₗₗₑₗₑ₁

eVai_{omozigote}=2*eVaiₐₗₗₑₗₑ

eVai_{compound}=eVaiₐₗₗₑₗₑ_{₁}+eVaiₐₗₗₑₗₑ_{₂}

One information item to be provided as input for each subject is the set of phenotypes which characterizes it. Since determining the specific phenotypic traits of a subject can be complicated from a clinical point of view and in some cases the process can be influenced by the person performing the evaluation, the calculation of all the features related thereto has been implemented with the aim of mitigating the problem; in fact, the algorithms used take advantage of the structure of the Human Phenotype Ontology, which can be represented by a direct acyclic graph (DAG) in which the terms are connected through hierarchical relationships of the is-a type according to which a term represents a more specific and detailed instance of the parent term thereof (e.g., “Abnormality of limbs” is a more general term than “Abnormality of limb bone”). By adopting the ontology tree structure for the calculation of phenotypic features, the impact of choosing a very specific term instead of a more generic term (and vice versa) is minimal; this allows freeing the clinician from the responsibility of providing a specific and precise characterization of the patient's phenotypic traits.

Once the features to be used have been defined, the training procedure of the machine-learning algorithms is implemented, for example by means of the implementation option shown in FIG. 2.

The training step aims to extract a general rule which associates the input with the desired output, learning such a rule (which is specified in the parameters of each model) from a dataset of examples the class of which is known a priori.

Once the classifiers have been trained, a test step on examples independent of the training dataset is necessary, in order to evaluate the performance of the algorithms and validate the methodology.

In the example shown here, the training set on which to train the ML classifiers was generated from two publicly available resources, mentioned below.
1. The examples of so-called positive (or pathogenic) inputs represent combinations of digenic variants known to be causative of a digenic disease, which in turn is characterized by a specific set of phenotypes; such examples are collected in the online DIDA database (Digenic Disease Database).
2. The so-called negative (or benign) input examples consist of combinations of digenic variants generated from the resources of the 1000 Genome Project containing the sequencing data and therefore the variants of healthy subjects; for this reason, it can be assumed that whatever the phenotype associated with such digenic combinations, there is no basis that such an instance can be considered causative of such phenotypic traits.

Starting from the set of examples described above, the matrix of features describing the training set was calculated; having a dataset strongly unbalanced towards negative cases (990 negative vs 255 positive), in addition to the training of the classifiers with the original data, two different data resampling strategies were implemented, aimed at obtaining a balanced distribution between the two classes:
1. Oversampling is a technique which randomly resamples the examples of the minority class.
2. “SMOTE” is a methodology which generates “summary” examples very similar to the examples of the minority class from the features matrix.

Having a mixed dataset (continuous, discrete and binary features), six machine-learning (ML) algorithms were finally chosen to process such a data type. In a method embodiment, all of the following classifiers (each of which is known per se) are used:
Decision tree;
Random Forest;
Adaboost;
Gradient Boosting;
Logistic Regression;
Multilinear Perceptron.

For each classifier and for each balancing technique, a robust iterative algorithm was used to determine the optimal set of hyperparameters, i.e., the one which allow better performance on the test set. The training step was lastly refined through the optimization of the decision threshold, a parameter used by each classifier to discriminate an example as positive or negative once a class probability value associated therewith has been calculated.

In this example, the training module generates a total of 18 classifiers (6 machine learning algorithms with 3 different balances of the training dataset); such classifiers can finally be used to classify new examples the possible pathogenicity on a digenic basis of which is to be determined.

Hyperparameters are the classifier parameters which directly control the training process and have a significant impact on the performance of the classifier itself in terms of speed and learning quality. The selection of the hyperparameter search space is part of the model selection step. The optimization consists in finding a tuple of hyperparameters which produces an optimized model which minimizes a predefined loss function on the test data and represents a crucial step for maximizing the predictive efficiency of the classifier. The strategy adopted for this purpose in the present example of the method has been chosen with the aim of finding a valid compromise between computational cost and efficiency and consists of a random search in the predefined space of the parameters.

In the state of the art, this technique is among the most efficient, as it involves an exhaustive search at the same time limited to the candidate parameters only which provide an effective improvement in learning. The search for the best set of parameters is performed using Scikit-Learn's RandomSearchCV function. For the continuous hyperparameters, a search space described by a uniform or exponential distribution can be provided. For the discrete parameters, a list of sampled values with a reasonable sampling step is provided. For the categorical parameters, all possible values are indicated. The random search is performed on 200 iterations, since such a value has proved to be an excellent compromise between performance and computational times. RandomSearchCV offers the possibility of using all the processors of the machine to increase the calculation speed. The times are dependent on the type of classifier for which the hyperparametric tuning occurs.

Through the application of the Stratified k-fold Cross Validation technique, each iteration, characterized by a specific combination of parameters, is trained on k-1 fold and tested on the remaining fold. In particular, a k=5 Cross Validation is performed, with a view to optimizing the timing and based on the number of datasets available. The stratified Cross Validation is so referred to since it ensures that, in each fold, the same proportions of the two classes in the whole training set are maintained. It is particularly suitable in the case of unbalanced data, as it ensures undistorted learning with respect to the majority class. If applied to a balanced dataset, it can be assumed that it has no effect, the simple k-fold Cross Validation being probabilistically equivalent to the stratified version in the case of a balanced training dataset.

Returning now to the prediction model, a flow diagram of the prediction model adopted in an embodiment of the present method is shown in FIG. 1; it takes as input on the one hand the list of individual variants identified in a patient with associated annotations in terms of gene involved and pathogenicity score, on the other hand a set of phenotypic terms articulated according to the HPO ontology.

Once the combinations of variants have been generated as explained above, the features matrix associated with the input examples is calculated; such a matrix is processed by the machine-learning algorithms which allow each example to be classified as pathogenic or benign.

As can be seen, the objects of the present invention as previously indicated are fully achieved by the method described above by virtue of the features disclosed above in detail. In fact, the method allows improving the accuracy in the classification of digenic variants, based on an approach which integrates predictive techniques based on machine learning and information on the patient's phenotype.

It is worth noting that the “digenic” approach which characterizes the present invention, which considers pathogenicity and phenotypic similarity of combinations of variants on two genes, is radically different from previous solutions based on a “monogenic” approach. In these, the impact of each variant on the phenotype is evaluated individually and these individual features are then combined into a “digenic” score. The method proposed herein also uses different information (such as the biological distance and the phenotypic similarity index), which capture the existing digenic relationships between the variants, which cannot be calculated from the “monogenic” information alone. With respect to previous solutions, the present method thus not only describes a combination of variants on two genes as the sum of the individually calculated scores for each variant, but also models the existing relationships between the variants and includes them in the score.

In order to meet contingent needs, those skilled in the art may make changes and adaptations to the embodiments of the method described above or can replace elements with others which are functionally equivalent, without departing from the scope of the following claims. All the features described above as belonging to a possible embodiment may be implemented irrespective of the other embodiments described.

1. A computer implemented method to determine pathogenicity of combinations of digenic or oligogenic variants, in relation to a disease, comprising the steps of:
defining a set of variants, the pathogenicity of which must be determined, wherein said variants refer to mutations present in one or both alleles of a respective gene of at least two genes, each of the genes being associated with two respective alleles;
determining situations which can occur, regarding the presence or absence of said variants in the alleles of said at least two genes, wherein each situation is associated with a respective combination in which each variant is present in a respective subset of alleles, among all possible subsets of alleles of all the genes considered, or each variant is present in all the alleles of all the genes considered;
for each of said defined situations, or for each combination and for each gene, calculating a pathogenicity index or score, adapted to estimate how much the one or more respective variants modify functioning of the respective gene;
describing phenotypic traits of a patient, by standardized phenotypic terms, comprising standardized information adapted to describe phenotypic anomalies found in the patient;
calculating or preparing input information for the pathogenicity determination, said input information comprising:
gene-phenotype association features, calculated individually for each of the genes considered, and adapted to measure how much said phenotypic traits of the patient are superimposable to phenotypes already known to be associated with the single gene;
digenicity or oligogenicity features, calculated for each of said combinations of genes, adapted to capture interaction between the genes forming each combination;
a priori property features of the genes, calculated for each of said genes considered;
variant-related features, calculated, for each gene considered, based on said pathogenicity indices or scores calculated in relation to all the combinations considered;
providing said input information for the pathogenicity determination to at least one trained algorithm;
processing said input information for the pathogenicity determination by the at least trained algorithm,
wherein said trained algorithm is an algorithm trained by artificial intelligence and/or machine learning techniques,
wherein said algorithm is trained in a preliminary training step, based on a training dataset of known cases, providing said input information calculated for each of known cases to algorithm to be trained, and training the algorithm based on the knowledge of the pathogenicity/benignity of the respective known cases;
obtaining output information from the trained algorithm, representing the pathogenicity of each of the combinations of variants or mutations considered.

2. A method according to claim 1, adapted to determine the pathogenicity of digenic variants, wherein:
in said step of determining possible situations, the identifiable situations comprise, for each of the variants considered and for each of the two genes, a situation of simple heterozygosity, in which the variant is present in only one of the two alleles, and a situation of compound heterozygosity, in which two different variants are present and each of the two variants is present on a respective allele, and a further combination of homozygosity, in which a same variant is present in both alleles of the gene;
and wherein:
said gene-phenotype association features comprise four features, two for each of the two genes;
said digenicity or oligogenicity features comprise two digenicity features calculated with reference to the two genes;
said a priori property features of the genes comprise six features, three for each gene;
said variant-related features comprise two features, one for each of the two genes, calculated, for each gene considered, as a combination of the pathogenicity scores of the variant(s) referring to said gene.

3. A method according to claim 1, wherein the step of describing phenotypic traits of a patient comprises:
describing phenotypic traits through terms deriving from an ontology which provides a standardized vocabulary of the phenotypic anomalies found in human diseases, or through the HPO terms, deriving from the resource Human Phenotype Ontology.

4. A method according to claim 3, wherein the description of phenotypic traits by HPO is represented by a direct acyclic graph.

5. A method according to claim 1, comprising, after the step of calculating, for each variant identified in the patient, a pathogenicity index or score, and after the step of describing a patient's phenotypic traits, the following further steps:
providing a list of defined variants, with the respective genes and respective calculated pathogenicity indices, to a first pre-processing algorithm, configured to generate a list of possible combinations;
processing said list of possible combinations, generated by the first pre-processing algorithm, as well as said phenotypic traits of the patient, in the terms described, by a second pre-processing algorithm, to calculate or prepare input information for the pathogenicity determination.

6. A method according to claim 1, wherein said gene-phenotype association features comprise, for each of the genes considered:
an index or measure of similarity between the set of standardized phenotypic terms describing the patient and the set of standardized phenotypic terms associated with the gene; and
a probability of association between the single mutated gene and the set of phenotypes which describes the patient using gene expression data, or starting from a transcriptomics analysis.

7. A method according to claim 1, wherein said digenicity or oligogenicity features, for each combination of genes, comprises:
a measure or index of biological distance, which represents how much the proteins produced by the two genes are involved or not involved in same biological processes with a certain degree of interaction, or which represents a degree of functional association between two or more genes, articulated according to a series of levels of evidence; and
a measure or index of similarity between the phenotypic sets associated with the genes, regardless of the phenotypic traits describing the patient.

8. A method according to claim 1, wherein said a priori property features of the genes comprise, for each gene:
one or more measures or indices representing how sensitive each gene is or is not to a gene dosage.

9. A method according to claim 1, comprising, before using said trained algorithms, a further preliminary training step, carried out based on two subsets of said training dataset containing data referring to known cases,
a first subset being used as a training database, or training set and a second subset being used as a validation database or test set.

10. A method according to claim 8, wherein the preliminary training step comprises training a plurality of trained algorithms, or classifiers, and evaluating performance of each classifier.

11. A method according to claim 10, further comprising the step of selecting a subset of trained algorithms, for the processing, based on said preliminary performance evaluation.

12. A method according to claim 9, wherein, once the preliminary training step has been completed, the step of processing the input information for the pathogenicity determination comprises processing the information by all the trained algorithms or classifiers used in the training, or by the trained algorithms or classifiers selected during the training.

13. A method according to claim 10, wherein said trained algorithms or classifiers comprise one or more of the following:
Random Forest, AdaBoost, Gradient Boosting, Logistic Regression, Multi-layer Perceptron, Decision Tree.

14. A method according to claim 9, wherein said first training subset, or training set is generated from two data resources:
a first data resource comprising examples of positive inputs, which represent combinations of digenic variants known to cause a digenic disease, in turn characterized by a specific set of phenotypes;
a second data resource comprising examples of negative inputs, consisting of combinations of digenic variants of healthy subjects.

15. A method according to claim 14, comprising the further step of balancing the data, in the case of data resources unbalanced towards negative cases, to obtain a balanced distribution between the two classes,
wherein said data balancing step is performed using the oversampling methodology or by SMOTE methodology.

16. A method according to claim 1, wherein said output information comprises an estimated pathogenicity probability of at least one combination of digenic or oligogenic variants considered, or of a plurality of combinations of variants among the digenic or oligogenic variants considered, or of all the combinations of digenic or oligogenic variants considered.

17. A method according to claim 16, wherein the output information further comprises, for each combination of digenic or oligogenic variants, a binary result representative of whether the combination of digenic or oligogenic variants is pathogenic or benign, obtained by comparing pathogenic probability estimated for the digenic or oligogenic variant with a respective threshold, associated with the combination of digenic or oligogenic variants.

18. A method according to claim 1, wherein said output information comprises identification of a most relevant oligogenic combination of variants, or a best digenic pair of variants, from a set of combinations of oligogenic or digenic variants considered, which has a most relevant correlation with a set of phenotypes describing the patient.
